(19) United States
(12) Patent Application Publication
(10) Pub. No.: US 2013/0236428 A1
(43) Pub. Date: Sep. 12, 2013

(54) Reprogramming of Aged Adult Stem Cells

(71) Applicant: Vincent C. Giampapa, Montclair, NJ (US)

(72) Inventor: Vincent C. Giampapa, Montclair, NJ (US)

(21) Appl. No.: 13/785,691

(22) Filed: Mar. 5, 2013

Related U.S. Application Data
(60) Provisional application No. 61/608,480, filed on Mar. 8, 2012.

(51) Int. Cl. C12N 5/071

(52) U.S. Cl. CPC C12N 5/0696
USPC 424/93.7

(57) ABSTRACT

Reprogramming of mammalian stem cells including aged human adult stem cells of all types with young adult stem cell's supernatant-intracellular matrix, bioactive lipids and or microvesicules in a single step or as a secondary two step process using oocyte supernatant, its intracellular matrix and/or cellular components are disclosed to accomplish a gene erasure and reprogramming. This invention focuses on reprogramming and/or reactivating genes that are active and involved in youthful adult stem cell function, within aged adult stem cells that have been previously collected by and/or stored for patients who are 40 years and older. The process is accomplished by using the natural unaltered young adult stem cell fluid and its cellular components.

### REFERENCE TO RELATED APPLICATION

This application claims the benefit under 35 USC 119(e) of the provisional patent application Ser. No. 61/608,480, filed Mar. 8, 2012, which is hereby incorporated by reference in its entirety.

### BACKGROUND OF THE INVENTION

Adult stem cells normally age with each decade and decrease in number and functional quality. This contributes to aging and loss of function of the human body as well as contributes to the diseases of aging like diabetes, decreased immune function arthritis, and cardiovascular disease, and other age-related disease processes

The instant invention is an improvement in the field because no methods exists to improve the function of previously collected and stored old adult stem cells using unaltered young stem cell fluid and cellular components, with or without, oocyte of fetal component retreatment.

This invention focuses on reprogramming the genes involved in youthful stem cell function within cells that have been previously collected and stored using a number of techniques including apheresis, bone marrow aspiration or other accepted collection methods. The cells of persons who are 40 years and older have multiple functional-cellular defects, due to the cellular aging process, that can be corrected by using natural unaltered young cell fluid and cellular components, of the young adult stem cells.

The relevant prior art, as known to the inventor, includes a May 2011, publication in FASEB. 5-25. 1474-1485 (2011) by Sun et al, entitled Rescuing Replication and Osteogenis of Aged Misenchynal Stem Cells by Exposure to Young Extracellular Matrix; PCT Publication WO 2004/04855 A1 to Millar et al, entitled Restoration of Methylation States in Cells; and PCT Publication WO 2007/016245 A2 to Fitzsimmons et al, entitled Reprogramming of Adult or Neonic Stem Cells and Methods of Use.

### SUMMARY OF THE INVENTION

A method of aged adult stem cell (AASC) reprogramming comprising the steps of: (a) collecting young adult stem cells (YASC) from the blood of a human donor, using aphoresis or like means; (b) using normal diffusion, cell lysis, or like means to at least partially dissolve membranes of said YASC to release an intracellular matrix (ICM) thereof; (c) applying a supernatant of said YASC to a culture of AASC to be reprogrammed; and (d) following exposure of said AASC to said supernatant for a bioactively sufficient period, infusing such an exposed AASC to the donor thereof, either with or without cell expansion.

Reprogramming of mammalian and/or aged human adult stem cells with young adult stem cell's supernatant-intracellular matrix, bioactive lipids and/or microvesicules in a single step or as a secondary two or three step process using oocyte supernatant, its intracellular matrix and/or cellular components are disclosed to accomplish a gene erasure and reprogramming. This invention focuses on reprogramming and/or reactivating genes that are active and involved in youthful adult stem cell function, within aged adult stem cells that have been previously collected by and/or stored for patients who are 40 years old or more. The process is accomplished by using the natural unaltered young adult stem cell fluid and by aphoresis of its cellular components.

It is an object of the invention to rejuvenate aged adult stem cells by exposing the same to the intracellular matrix of one or more of youth adult stem cells, of oocytes and/or of fetal factors.

The above and yet other objects and advantages of the present invention will become apparent from the hereinafter set forth Brief Description of the Drawings and Detailed Description of the Invention.

### BRIEF DESCRIPTION OF THE DRAWINGS

FIG. 1 is a conceptual flow diagram of the inventive method showing the use of the supernatant of young adult cells to reprogram aged adult stem cells and, optionally, the use of oocytes and fetal factors therewith.

### DETAILED DESCRIPTION OF THE INVENTION

As stated above, adult stem cells normally age with each decade and decrease in number and functional quality. This contributes to the human aging process and loss of function of the human organs and body. Such aging occurs at an epigenetic level in which important promoter regions of DNA progressively turn-off or cease to function optimally. The invention claimed here solves this problem by enhancing the function of older adult stem cells by exposing the AASC to the cellular products and cell components of youthful stems, oocytes and/or fetal factors, each of which have different cellular products and cell membrane components.

Reprogramming of adult stem cells with a young stem cell's supernatant or its ICM or with oocyte supernatant or its ICM, reprograms and reactivates promoter regions of aged genes to a more youthful profile that are involved in maintaining youthful stem cell function. This occurs by “turning off” certain age-related genes and at the same time “turning back on” other genes. This results in a more efficient body cell replacement, turnover and improved quality of health, at the cellular and higher tissue and body levels.

The claimed invention differs from what currently exists. The present techniques involve use of viral vectors and gene splicing technologies, which are fraught with potential problems including but not limited to abnormal cell growth, this including multiple steps where the reprogramming cannot be reproducible on a regular basis. This process is also expensive and labor intensive.

There presently does not exist any effective clinical method of improving previously collected and stored human old adult stem sells and their function, using the ICM of young stem cell or oocyte's ICM so that such cells can be used to reduce human aging and improve the quality of health as humans grow older. That is, there presently does not exist any clinical method of enhancing previously collected and stored old adult stem cells and their function, using the ICM of young adult stem cells or oocyte's supernatant or ICM so such cells can be used to improve human aging and quality of health without use of complicated cellular manipulation. This permits the stem cells to be used for clinical intervention as a means of maintaining health and normal function in aging humans.

The past systems of stem cell manipulation did not focus on gene reprogramming of specific genes or groups of genes involved with youthful stem cell function, by using young cell intra-cellular fluid and components, either with or without oocyte cell fluid or cell components.

This invention focuses on reprogramming the genes involved in youthful adult stem cell function within the cells that have been previously collected and stored for patients who are 40 years and older, by using the natural unaltered young cell fluid and its components.

The Invention Discussed Relates to:

Individual cell components which include but are not limited to

amino acids

bioactive lipids

chemokines

culture media

DNA

hormonal compounds

iRNA

microvesicles

micro-rna

mRNA

old stem cells

oocytes

oocyte cell fluid components

peptides

polypeptides

RNA

transcription factors

various growth factors

young stem cells

### Relationship Between Cellular Components:

The invention's active reprogramming components are contained within the young stem cell's cellular components as a natural mixture of the intra cellular matrix (ICM). The microvesicles are part of the cellular membrane itself.

The first step necessary in this method is to place previously collected old adult stem cells, after using an aphoresis collection process with neupogen as a mobilizing agent, on a standard culture media or a cell culture dish which is required to expose the old stem cells to the intra cellular and membrane components of the young adult stem cell. The young adult stem cell components may be released by any number of standard cell rupture (lysis) techniques or by simple diffusion. In this fashion the soluble factors and the cell membrane components and microvesicles are able to diffuse or transfer themselves passively through the old stem cell membrane and initiate the gene reprogramming effects on appropriate promoter regions of DNA of the aged stem cells.

Principles of The Invention

The reprogramming of the old stem cell genes occurs through direct exposure of the old stem cells to young stem cell intracellular and membrane components. The cells to be treated comprise old cells placed on a culture dish and separated from the young cells by a permeable membrane that allows the young cell's soluble factors and bioactive lipids of cell membrane to cross over to and be transferred directly into and through the cell membrane of the old stem cell. Once this has occurred the promoter regions of the genes of the old stem cell are re-programmed by the young cellular components after an adequate time of exposure to the young cellular elements. most of the gene reprogramming occurs at the level of the chromosome, the epigenetic level, and through the processes of methylation, acetelyation and/or phosphorylation. This process can be completed as described in Step 1, or a more complete reprogramming can be accomplished in two or three steps.

If a more complete “gene reprogramming” is desired then the old stem cells are first exposed to oocytes' intracellular fluid components via the same process as described above, and complete what can be termed “gene imprinting erasure”. The “erasure process” is accomplished because oocytes contain different intracellular soluble factors that do not strictly reprogram but “erase” certain methylation patterns and other epigenetic markers of old cells. In this more complete process the “genetically erased” old adult stem cells are treated with the other step described above where it is exposed to the young stem cell's supernatant, its soluble factors and cell membrane components (micro-vesicles), to reprogram its genes to a similar profile found in the young adult stem cell.

### Clinical Procedure

### Step One

In the complete process, the procedure can begin by placing the old adult stem cells in the presence of an oocyte and allowing the intracellular components from this group of cells to passively transfer through a dividing membrane and into the old stem cells. This removes some or all of the “genetic programming” or gene Imprinting in the old stem cells. After this process is completed, the original-old gene imprinting is removed or (erased) and the old stem cell genes are now ready to be reprogrammed.

### Step Two

This step involves placing the old stem cells with the “erased genes” into the presence of the young stem cell solution consisting of but not limited to the soluble factors and microvesicle biolipids, and allowing appropriate time for the old-cleaned genes to be reprogrammed by normally occurring diffusion of these elements thru the an old stem cell membrane. The diffusion process can be aided by a number of standard cell diffusion enhancement techniques. During this step in the process the cells are not expanded or multiplied. The elements in this invention that are optional are the oocyte, gene erasure step or fetal fluid exposure process and the exposure to their intracellular components, i.e., the gene cleaning or “gene imprinting erasure” step may be optional.

The young stem cell factors and bioactive lipids are necessary to interact with the genes of the older adult stem cells to effect the reprogramming process at the epigenetic level. For the invention to work more efficiently, the factors and bioactive lipids could be taken from a genetically related human subject like a son or daughter that is younger, but this is not a necessary factor to render the process effective. markers that are used to identify and confirm the identity of young adult stem cells are beta-galactosidase, telemerase and colony forming units. These markers and factors may be measured prior to exposure to the older cells to be reprogrammed and can be premeasured or remeasured after the regrogramming process to document its effectiveness.

The process described above can be further enhanced by the use of fetal factors and therein biosoluble lipids which can be obtained from umbilical cord stem cells or the amniotic fluid related to birth. Such cells include mesenchymal stem cells, adipose derived stem cells, stromal cells, skeletal muscle stem cells, neural stem cell, cardiac stem cells and amniotic fluid cells.

### How to Use the Invention

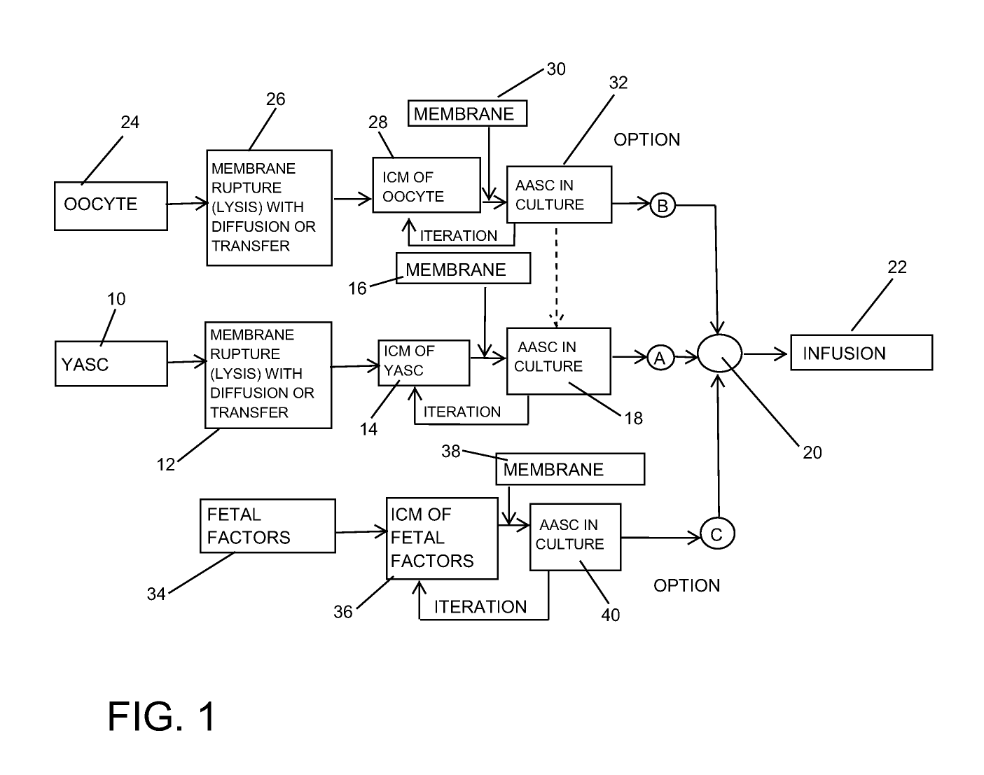

With reference to FIG. 1, there is shown in flow diagram form the present method of age adult cell reprogramming, illustrating therein the primary method involving the use of youthful adult stem cells as well as two optional or enhancement methods thereof which, respectively, entail the use of oocytes and fetal factors or amniotic fluid cells. More particularly, looking at the central row in FIG. 1, the process begins with the collection of youthful stem cells 10 by any of a number of accepted stem cell collection techniques including but not limited to apheresis processes. The collected young adult stem cells (YASC) are then, at step 2, subjected to membrane rupture, known as lysis, using any of a number of known processes such as the use of biological detergents or simple diffusion. Such lysis enables diffusion or transfer of the intracellular elements of the stem cells, known as the intracellular matrix (ICM) as step 14. Therefrom the ICM passes through a membrane 16, the purpose of which is to allow selective filtration of the desired intracellular components or membrane components of the young stem cells. The ICM components passing through membrane 16 then are exposed to a culture of the aged adult stem cells to be reprogrammed, shown at step 18. If this is the sole strategy employed in a given AASC reprogramming project, the culture of step 18 is collected at Step A, concentrated at step 20 and infused, at step 22, into the donor of the AASC. Cell expansion can then be accomplished at this stage prior to using the cells for therapy.

In the event that an oocyte 24 is employed, prior to above-described steps 10 to 18, for purposes of “cleaning” of age-related characteristics of the AASC, the oocyte is then subject to lysis using a method suitable to the membrane thereof, this shown at step 26. The ICM of the oocyte is them employed at step 28 and passes through membrane 30 to form a culture 32 in combination with the AASC. Both steps 18 above and said step 32 may be reiterated relative to steps 14 and 28 respectively several times in order to assure maximum potency of the resultant concentrate B.

As a further option, intended to enhance the effectiveness of the present method, fetal factors 34 may be employed from which the ICM 36 are extracted. These then pass through membrane 38 and are added to the AASC in culture at step 40. The concentrate thereof (indicated by letter C) is mixed at the aggregate concentrate step 20 and then infused into the donor.

As may be seen in FIG. 1, old adult stem cells are exposed to the supernatant of young adult stem cell (Step 18) on a standard cell culture media, in vitro, with or without use of a semipermeable membrane 16 between them. The old adult stem cells are then left in contact with the young adult stem cell's supernatant (containing the soluble compounds and micro vesicles) for a specific amount of time and/or thru serial exposures, to avoid dilution by the old stem cell components. This process allows the old stem cells genes to be “reprogrammed,” that is, promoter regions of some genes are “turned off” while others are “turned on.” This process results in a more youthful gene profile resulting in a functionally “reprogrammed old adult stem cell.”

The inventive method can also produce a new class of cell products for intravenous infusion for:

1) treating the aging process in general,
2) treating immune deficiencies,
3) treating Aids patients,
4) treating a general class of age related disease.

The invention can be used topically for:

1) wound healing with these re programmed cells,
2) treating aging skin to prevent and remove wrinkles,
3) treating of skin cancers.

The reprogrammed cells can be used in the experimental lab studies for in vitro cell studies with other stem cells as a comparison to see the effects of other gene expression modifiers.

While there has been shown and described above the preferred embodiment of the instant invention it is to be appreciated that the invention may be embodied otherwise than is herein specifically shown and described and that, within said embodiment, certain changes may be made in the form and arrangement of the parts without departing from the underlying ideas or principles of this invention as set forth in the Claims appended herewith.

I claim:

1. A method of reprogramming aged human or animal adult stem cells (AASC) of all types, comprising the steps of:
(a) collecting young adult stem cells (YASC) from the blood of a donor, using aphesis or like means;
(b) using cell lysis or simple diffusion to at least partially dissolve membranes of said YASC to release an intracellular matrix (ICM) thereof;
(c) applying a supernatant of said ICM of said YASC to a culture of AASC to be reprogrammed; and
(d) following exposure of said AASC to said supernatant for a bioactively sufficient period, infusing such an exposed AASC to the donor thereof.

2. The method as recited in claim 1, further comprising:
re-iterating said Steps (b) and (c).

3. The method as recited in claim 1, further comprising:
separating said YASC from said supernatant of said YASC by use of a membrane and permitting YASC soluble factors and related components to pass therethrough prior to application of said Step (c).

4. The method as recited in claim 3, further comprising:
re-iterating said Steps (b) and (c).

5. The method as recited in claim 1, further comprising the steps of:
(e) prior to said Step (a), collecting oocytes biologically compatible with said AASC to be re-programmed;
(f) using lysis or like means to dissolve or remove membranes of said oocytes to remove the ICM thereof; and
(g) applying said ICM of said oocytes to said culture of said AASC.

6. The method as recited in claim 5, further comprising the steps of:
re-iterating said Steps (b) and (c).

7. The method as recited in claim 5, further comprising:
separating said YASC from said supernatant of said YASC by use of a membrane and permitting YASC soluble factors and related components to pass therethrough prior to application of said Step (c) in which promoter regions of the ASSC are reprogrammed by promoter regions of the YASC supernatant,

8. The method as recited in claim 7, in which said oocyte ICM affects different promoter regions which are reprogrammed which are the more primordial nature.

9. The method as recited in claim 1, further comprising:
applying fetal cell products or by-products together with or after said Step (c).

10. The method as recited in claim 5, further comprising the step of:
applying fetal cell products or by-products together with or after said Step (c).
